(19) United States
(12) Patent Application Publication
(10) Pub. No.: US 2021/0353213 A1
(43) Pub. Date: Nov. 18, 2021

(54) SYSTEM AND METHOD OF WOUND ASSESSMENT AND TREATMENT

(71) Applicant: MyWoundDoctor, LLC, Nashville, TN (US)

(72) Inventors: Daniel F. Heneghan, Wilmington, NC (US); Nicholas E. Sieveking, Franklin, TN (US); David E. Owen, Concord, MA (US)

(73) Assignee: MyWoundDoctor, LLC, Nashville, TN (US)

(21) Appl. No.: 16/875,428

(22) Filed: May 15, 2020

(51) Int. Cl. A61B 5/00; G16H 10/60; G16H 70/60; G16H 50/70; G16H 50/20; G16H 15/00; G16H 50/50; G16H 40/20; G16H 20/10; G06Q 10/08; A61B 5/107; G06T 7/00

(52) U.S. Cl. CPC A61B 5/445; G06T 2207/30088; G16H 70/60; G16H 50/70; G16H 50/20; G16H 15/00; G16H 50/50; G16H 40/20; G16H 20/10; G06Q 10/083; A61B 5/0077; A61B 5/681; A61B 5/6898; A61B 5/0013; A61B 5/0022; A61B 5/7267; A61B 5/4833; A61B 5/7435; A61B 5/7475; A61B 5/1072; A61B 5/7246; A61B 5/4848; A61B 5/4842; G06T 7/0012; A61B 2576/02; G16H 10/60

(57) ABSTRACT

The presently disclosed subject matter is generally directed to an improved system for assessing and treating a wound. Specifically, a wound is identified in a patient by uploading or entering information about the wound and/or patient (e.g., type of wound, wound location, patient history). The wound image is captured, such as through an imaging device (e.g., phone, tablet, computer) and uploaded to the system. The image is then reviewed by a professional, such as a medical professional that specializes in treating a variety of wounds. The disclosed system uses an algorithmic approach to develop a systematic method to integrate selected data about the patient and deliver clinical care suggestions immediately to the patient or caregiver.

### CROSS-REFERENCE TO RELATED APPLICATIONS

The current application claims priority to U.S. Provisional Patent Application No. 62/847,227, filed May 16, 2019, the entire content of which is hereby incorporated by reference.

### TECHNICAL FIELD

The presently disclosed subject matter is generally directed to system and method that can be used to assess and treat a wound.

### BACKGROUND

Chronic and complex wounds (e.g., venous ulcers, diabetic ulcers, pressure ulcers, surgical wounds) affect millions of patients in the United States alone. As a result, billions of dollars are spent on the treatment of chronic wounds in the United States annually. The number of chronic wounds continues to grow as the population ages and due to the rising incidence of diabetes and obesity. Healing and chronic non-healing wounds are associated with a number of biological tissue changes including inflammation, proliferation, remodeling of connective tissues, bacterial infection, and mortality rates that are equivalent to cardiac-related deaths. Wound care has therefore become a major clinical challenge. Advances in medical technology have brought new treatments to the various wound types. However, the new technologies lack accurate and objective assessment of wounds and the wound healing progress. For example, in the clinical setting, wound depth, volume, area, circumference, and classification are not accurately determined. Specifically, these measurements are typically performed manually with a ruler, transparency tracing, and Q-tips for depth. After the wound is inspected, the health care provider determines the materials, sizes and shapes of wound dressings by selecting from an available inventory on hand. If a particular dressing material is not available, the practitioner must select an alternative, which may be less than satisfactory. It would therefore be beneficial to provide a system and method that overcomes the shortcomings of the prior art, providing for improved wound assessment and treatment.

### SUMMARY

In some embodiments, the presently disclosed subject matter is directed to a method for assessing a patient wound and providing a treatment plan. Particularly, the method comprises inputting data into a database, wherein the data includes patient medical history information and an image of the wound. The method includes correlating the data to a unique code to assess the wound and determining qualification for treatment based on the assessment of the wound. The method includes retrieving and storing collected data relating to historical wound assessment to provide a treatment plan for the wound and tracking and reporting progress of the treatment of the wound. The disclosed method facilitates lowest skilled care. The phrase “facilitates lowest skilled care” refers to allowing wound care treatment and/or assessment by a low skilled care worker, such as a nurses aid, patient caretaker (e.g., spouse), as well as skilled care workers (doctors and/or wound care specialists).

In some embodiments, the image of the wound is input into the database by uploading from an imaging device.

In some embodiments, the imaging device is selected from a camera-enabled mobile phone, smart phone, laptop computer, notebook computer, desktop computer, personal digital assistant, MP3 player, camera, ager. portable gaming device, electronic watch, portable music player, dedicated hardware computing device, or combinations thereof.

In some embodiments, the wound assessment comprises a determination of one or more of the following: patient medical history, wound exudate, wound odor, wound secondary infection, wound width, wound length, wound depth, and wound outcome.

In some embodiments, the database communicates with a mobile application for uploading, storing, and providing information related to the wound, the wound assessment, the treatment plan, and progress of the treatment of the wound.

In some embodiments, providing a treatment plan comprises a combined data-driven, algorithmic, and machine learning approach to deliver optimal clinical care protocols and related wound care supply needs and instructions.

In some embodiments, the wound is selected from one or more of a venous ulcer, skin ulcer, diabetic ulcer, pressure ulcer, surgical site infection, burn, or bite.

In some embodiments, facilitating lowest skilled care comprises providing treatment instructions and measuring compliance with treatment instructions.

In some embodiments, the treatment plan comprises instructions for healing the wound, procedures for healing the wound, formulary supplies, or combinations thereof.

In some embodiments, the method further comprises shipping formulary supplies directly to the patient based on the treatment plan.

In some embodiments, the unique code comprises patient demographic data, patient medical history, wound characteristics, or combinations thereof.

In some embodiments, the presently disclosed subject matter is directed to a system for assessing and treating wounds in a patient. Particularly, the system comprises an electronic device comprising a computer hardware, software, and display device for entering data about a patient into a database, wherein the data comprises patient medical history and an image of the wound. The computer hardware includes a storage device for receiving and storing information from the electronic device into the database. The system can comprise a mobile application associated with the electronic device and the storage device for correlating the data to a unique code to assess the wound and determine qualification for treatment based on the wound assessment. The mobile application retrieves and stores collected data relating to historical wound assessment from the database to provide a treatment plan for the wound and the mobile application provides for tracking and reporting progress of the treatment of the wound.

In some embodiments, the mobile application provides for the upload and display of an image of a wound.

In some embodiments, the image of the wound is input into the database by uploading from an imaging device comprising the mobile application.

In some embodiments, the imaging device is selected from a camera-enabled mobile phone, smart phone, laptop computer, notebook computer, desktop computer, personal digital assistant, MP3 player, camera, ager, portable gaming device, electronic watch, portable music player, dedicated hardware computing device, or combinations thereof.

In some embodiments, the wound assessment comprises a determination of one or more of the following: patient medical history, wound exudate, wound odor, wound secondary infection, wound width, wound length, wound depth, and wound outcome.

In some embodiments, providing a treatment plan comprises a combined data-driven, algorithmic, and machine learning approach to deliver optimal clinical care protocols and related wound care supply needs and instructions.

In some embodiments, the wound is selected from one or more of a venous ulcer, skin ulcer, diabetic ulcer, pressure ulcer, surgical site infection, burn, or bite.

In some embodiments, the mobile application coordinates shipping of formulary supplies directly to the patient based on the treatment plan.

In some embodiments, the unique code comprises patient demographic data, patient medical history, wound characteristics, or combinations thereof.

### BRIEF DESCRIPTION OF THE DRAWINGS

FIG. 1 is a flow chart illustrating a method of using a system to assess and treat a wound in accordance with some embodiments of the presently disclosed subject matter.

FIGS. 2a and 2b are a front plan views of imaging devices that can be used in accordance with some embodiments of the presently disclosed subject matter.

FIG. 3 is a chart illustrating the flow of information in accordance with some embodiments of the presently disclosed subject matter.

FIG. 4 is a schematic illustrating factors that can be included as code identifiers in accordance with some embodiments of the presently disclosed subject matter.

FIG. 5 is a schematic illustrating various considerations that are used in developing a treatment plan in accordance with some embodiments of the presently disclosed subject matter.

FIG. 6 is a flow chart illustrating the flow of information in accordance with some embodiments of the presently disclosed subject matter.

FIG. 7 is a flow chart illustrating a method of using a system to assess and treat a wound in accordance with some embodiments of the presently disclosed subject matter.

FIG. 8 is a flow chart illustrating a method of using a system to assess a wound in accordance with some embodiments of the presently disclosed subject matter.

FIG. 9 is a flow chart illustrating a method of using a system to assess and treat a wound in accordance with some embodiment of the presently disclosed subject matter.

FIGS. 10a-10h are photographs illustrating healing of a wound in accordance with some embodiments of the presently disclosed subject matter.

FIGS. 11a-11c are photographs illustrating healing of a wound in accordance with some embodiments of the presently disclosed subject matter.

FIGS. 12a-12c are photographs illustrating healing of a wound in accordance with some embodiments of the presently disclosed subject matter.

FIGS. 13a-13c are photographs illustrating healing of a wound in accordance with some embodiments of the presently disclosed subject matter.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a device” can include a plurality of such devices, and so forth. It will be further understood that the terms “comprises,” “comprising,” “includes,” and/or “including” when used herein specify the presence of stated features, integers, steps, operations, elements, and/or components, but do not preclude the presence or addition of one or more other features, integers, steps, operations, elements, components, and/or groups thereof.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

As used herein, the term “and/or” includes any and all combinations of one or more of the associated listed items.

The embodiments set forth below represent the necessary information to enable those skilled in the art to practice the embodiments and illustrate the best mode of practicing the embodiments. Upon reading the following description in light of the accompanying drawing figures, those skilled in the art will understand the concepts of the disclosure and will recognize applications of these concepts not particularly addressed herein. It should be understood that these concepts and applications fall within the scope of the disclosure and the accompanying claims.

The presently disclosed subject matter is directed to an improved method for assessing the status of a wound and providing treatment. The term “wound” as used herein broadly refers to a disruption of the normal continuity of structures caused by a physical (e.g., mechanical) force, a biological (e.g., thermic or actinic) force, and/or a chemical contact. Thus, typical wounds can include (but are not limited to) contusions, incisions, ulcers, stabs, lacerations, openings, penetrations, punctures, abrasions, grazes, burns, frostbites, corrosions, wounds caused by ripping, scratching, pressure, and biting, and other types of wounds. The disclosed system and method can automatically assess the wound directly from a digital image obtained using an imaging device (e.g., through a mobile application) and suggest an optimal treatment plan. The disclosed system is therefore intended to evaluate a patient wound and provide an immediate and personalized treatment plan, including wound treatment training video content to improve treatment plan compliance In some embodiments, computer-generated video content based on the treatment protocols can be integrated into the system.

As set forth in more detail below, the presently disclosed subject matter is generally directed to a method and system of effectively assessing and treating wounds. The disclosed system allows for effective, efficient, and streamlined treatment of wounds through the use of a telehealth platform. Advantageously, the disclosed system includes the use of a combined data-driven, algorithmic, and machine learning approach to deliver optional clinical care protocols and related wound care supply needs and instructions automatically at the point of care with a patient. As a result, expert clinical wound care assessment is delivered rapidly, which reduces medical costs and clinical inconsistencies typically associated with manual wound review and treatment. The disclosed system and method also improves the speed with which wound care treatment begins. In addition, the system allows the patient to remain in the lowest acuity care environment (and lowest cost), such as at home. The system reduces the frequency and total number of visits to a clinical care facility, thereby reducing travel requirements. Further, the system allows the care process to be enabled by one or more persons with a lower level of professional training, including the patient themselves (self-care). The system standardizes care, beginning with the option to pursue a least expensive but highest quality alternative. The system additionally emphasizes the timely resolution of open wounds (e.g., infection reduction). Accordingly, the disclosed system provides for efficient and accurate personalized wound care for a subject in need of treatment.

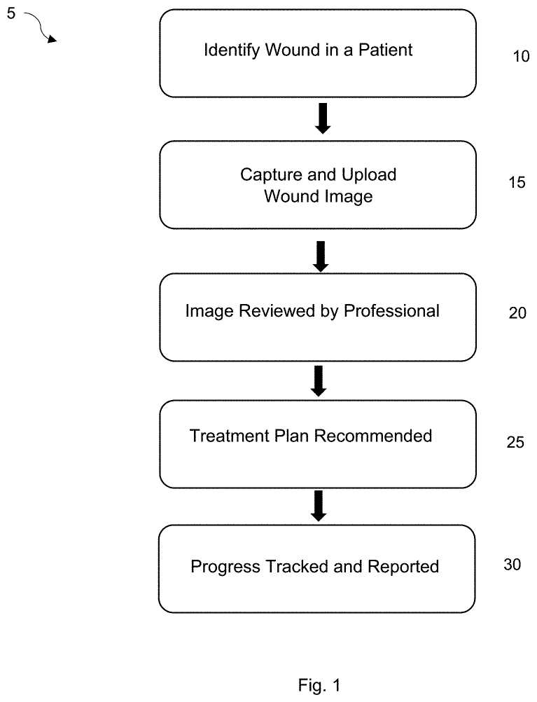

FIG. 1 illustrates one embodiment method 5 that can be used to assess and treat a wound. At step 10, a wound is identified in a patient. Typically, a care provider at a clinic, hospital, or lab performs any desired testing and observations related to the wound, and the results are input for storage in a medical database (e.g., an electronic medical record). An image of the wound is then captured, such as through an imaging device (e.g., phone, tablet, computer, digital camera) and uploaded to the disclosed system at step 15. At step 20, the image is reviewed by either a medical professional who specializes in treating a variety of wounds (Certified Wound Ostomy Nurse (CWON), Wound Care Certified (WCC), etc.) or a medical professional who supports patient care but who does not have certified wound care experience (e.g., Physician's Assistant, Registered Nurse, Medical Aide, etc.). The disclosed system uses an algorithmic and machine learning approach to integrate selected data about the patient and/or wound to deliver optional clinical care suggestions (e.g., protocols) based on the wound image identification and related treatment plans for each protocol that include wound care supply formulary items to be used for treating the patient wound, system-generated training video links to support each treatment step within the protocol, and an automated fulfillment and delivery process for the patient to receive the wound care supplies (e.g., home delivery, retail pharmacy pick-up, hospital-based supply delivery). In this way, a treatment plan and medical supplies necessary to heal the wound are recommended at step 25. The wound characteristics and healing progress are then tracked at step 30 using metrics, including tissue color, size of the wound, and clinical improvements over time. Advantageously, the disclosed system provides a streamlined approach for remote assessment and treatment of the wound. As a result, expert clinical wound care evaluation and treatment is delivered rapidly, which reduces medical costs and clinical inconsistencies typically associated with manual wound review and treatment. The disclosed system offers rural health care settings (e.g., critical access hospitals, urgent care centers) and home health care staff to support wound care treatment conveniently without wound specialist staffing requirements and also improves the speed with which wound care treatment begins. Accordingly, the disclosed system provides for efficient and accurate personalized wound care for a patient in need of treatment.

At step 10 of the disclosed method, a wound is identified in a patient. A variety of information about the wound and/or patient can be input into the disclosed system. Suitable information can include (but is not limited to) patient demographic and medical history, physical wound characteristics, and/or wound composition. Patient demographic and medical history information can include patient data, such as date of birth, gender, height, weight, contact information, ethnicity, blood type, nationality, treating clinician, insurance provider, allergies, major diagnoses, medical history, medications taken, medication history, family medical history, and the like. Physical wound characteristics can include any sensory features of the wound, such as (but not limited to) wound location, size, length, width, depth, edge observations, presence/absence of odor, pain level, and the like. Similarly, wound composition can include information related to the structure of the wound, such as wound colors, presence of seeping, degree of seeping, presence of infection, type of infection, degree of infection, level of healing, and the like. It should be appreciated that the input information is not limited and can include any information relevant to the patient or wound.

The input information can be manually entered by the patient and/or clinician into the system, such as through a system mobile application as discussed below. Alternatively or in addition, information can be automatically uploaded at step 10 through access to the patient's corresponding medical records (e.g., electronic medical records). In this way, information can be provided via a remote connection. It should be appreciated that the system can interact with a system user through interactive speech recognition that provides for structural answers based on a conversational user interface that responds to various user questions. The interactive speech recognition feature can respond/communicate with multiple language options. In this way, the system user can ask questions and receive responses and/or the system can ask questions to the system user to input information.

At step 15, one or more images of the wound are captured and uploaded to the disclosed system using an imaging device. The term “imaging device” includes any device that can capture an image of a wound, including (but not limited to) a camera-enabled mobile phone, smart phone, laptop computer, notebook computer, desktop computer, personal digital assistant (PDA), MP3 player, camera, handheld device, pager, portable gaming device, electronic watch, portable music player (e.g., an Apple iPod®), and/or dedicated hardware computing devices. In some embodiments, a secure imaging device (e.g., phone or camera) can be provided by the system to the user. The imaging device can operate using mobile operating systems such as iOS (from Apple Inc.) and Android (from Google Inc.), desktop operating systems such as OSX (from Apple Inc.) and Windows (from Microsoft Corp.), or any other known operating system or platform.

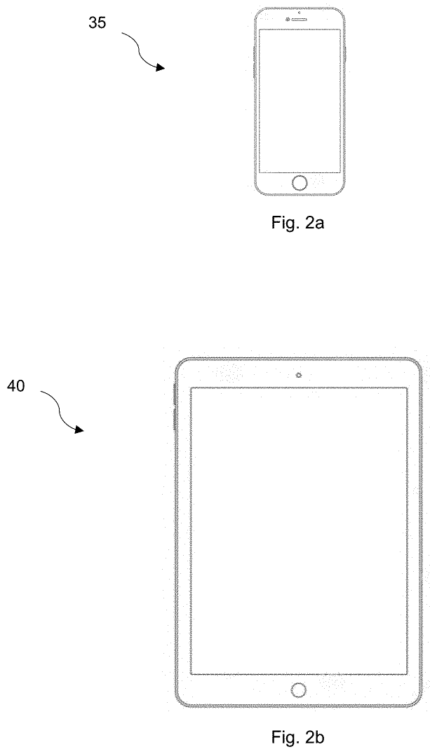

Two representative examples of imaging devices are illustrated in FIGS. 2a and 2b. Particularly, FIG. 2a illustrates smartphone 35 that can be used to download the corresponding system application and upload images and/or information to assess and treat a wound. FIG. 2b illustrates tablet computer 40 capable of imaging a wound, in addition to uploading and downloading patient, wound care, and treatment information.

In some embodiments, the imaging device can comprise a wireless transmission element for wirelessly transmitting the obtained images to the disclosed system. In some embodiments, the image can be uploaded directed through a system mobile application. Alternatively, in some embodiments the imaging device can transmit information using a wired connection as would be known in the art.

In some embodiments, the imaging device can include a corresponding system mobile application. The term “mobile application” refers to software used on a smart phone or other mobile computing device such as an Android®, iPhone®, BlackBerry® or iPad®. Mobile applications enable users to perform functions on their mobile devices “on-the-go” without the constraints associated with working at fixed locations, such as at home or in the office. It will readily be understood by a person of ordinary skill in the art that mobile applications are not limited to mobile phones but may also be used on laptops, home computers, and the like. In some embodiments, the disclosed software application can be integrated into any electronic medical record (EMR) application. Further, the disclosed application can be considered store and forward technology, as is known in the art.

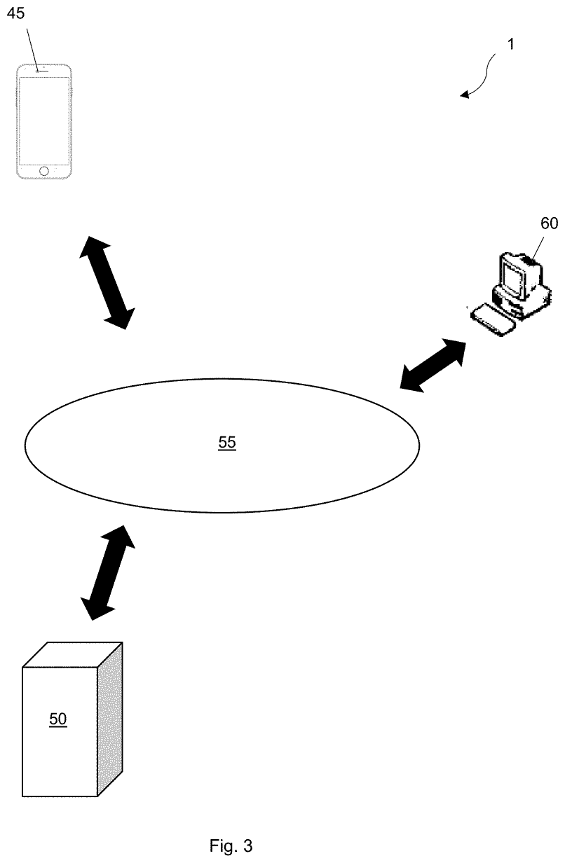

FIG. 3 is a schematic diagram of a basic system infrastructure that can be used in accordance with some embodiments of the presently disclosed subject matter. As shown, the basic hardware layout of system 1 can comprise imaging device 45 (e.g., smartphone, laptop, Alexa, HomePod®, etc.) for use at the location of a patient and a remote database or server 50 in communication with the wireless device via internet 55 or any other suitable communication medium. Internet access can be accomplished though any suitable connection systems including, for example, dial-up, DSL, high-speed digital network, LAN, WAN, WI-FI, cellular telephone, laser light transmission systems, satellite systems, infrared carrier systems, or nuclear- or bioparticle-powered information transmission systems Optionally, one or more analysis/diagnosis workstation(s) 60 also communicate with database 50 and/or wireless device(s) 45 via internet 55 or in another suitable manner. The system can include one or more processors, servers and databases, as well as suitable software for running the system, as would be known in the art.

The information and/or images are sent (e.g., via cellular communication) into a password-protected web server, i.e., database 50. At step 20, the data and wound images are reviewed via the internet by one or more members of a physician-led health care team connected to the patient via workstation 50 or through their own imaging device (e.g., smart phone). In some embodiments, the image can be reviewed on a secure telehealth console. The term “telehealth” refers to the acquisition of healthcare information remotely via telecommunications technology. The telecommunications equipment can include capturing, displaying, recording and/or transmitting diagnostic and/or treatment information, including remote control manipulation of devices and/or diagnostic audible information gathering tools (e.g. interactive kiosks, personal assistant devices (Alexa, Siri, Mini@Home®), etc). The server and the smart phones or other imaging devices can utilize the system software application to help guide accurate assessment and treatment of the wound.

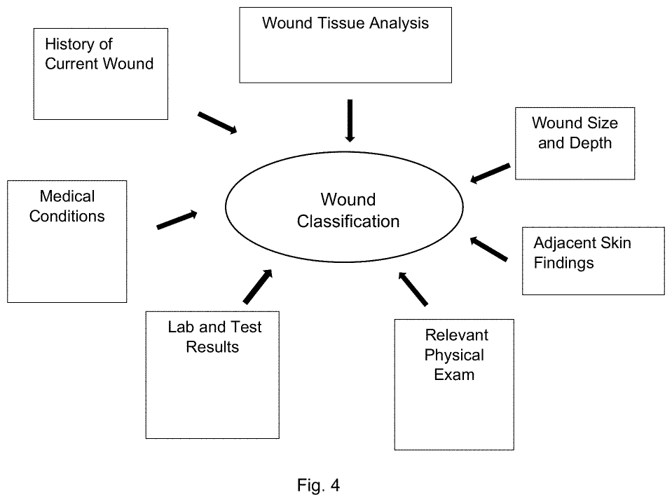

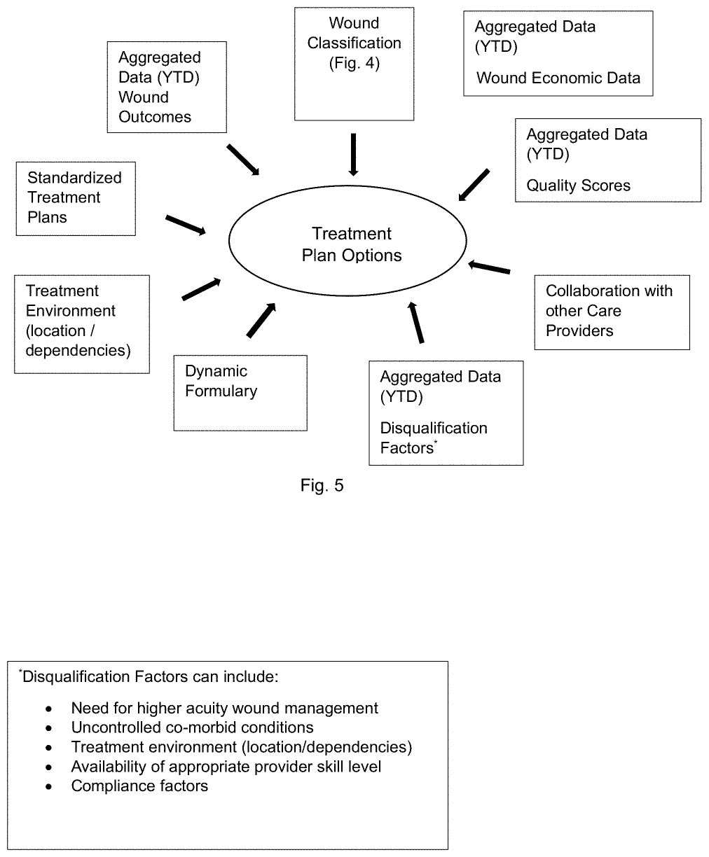

After the wound is captured and uploaded, one or more code identifiers can be assigned to the image and/or associated with the wound based at least in part on the historical patient demographic and/or medical history, as illustrated in FIG. 4. The relevant physical exam can include information such as year of birth, sex, ethnicity or race, available family and/or medical history, social and educational history, and/or geographic data. Representative medical history can include information such as medical conditions (e.g., previous injuries and/or wounds), history of the current wound (e.g., cause of the wound, age of the wound, etc.), lab and test results (e.g., diagnostic data such as MRI images, etc), and the like. Additional code identifiers can be assigned relative to one or more historical wound characteristics, such as (but not limited to) wound depth, wound length, wound width, wound odor, appearance of the wound edges, wound location, wound size, adjacent skin findings, wound tissue analysis, and the like.

For example, any of the wide variety of known standard administrative coding sets can be used, such as (but not limited to) ICD, HCPCS, CPT, LOINC, DRG, and/or MDS for PDPM LTC reimbursement, and OASIS for PDGM home health reimbursement. ICD (Internal Classification of Diseases) is the international standard diagnostic tool for epidemiology, health management, and clinical purposes. HCPCS (Healthcare Common Procedure Coding System) is a standardized code system that allows medical providers to submit healthcare claims to Medicare and other health insurances in a consistent and orderly manner. CPT (Current Procedural Terminology) codes are published by the American Medical Association and are the primary means of reporting the provision of medical services. LOINC (Logical Observation Identifiers Names and Codes) is a database and universal standard for identifying medical laboratory observations. DRG (Diagnosis Related Group) is a system used to classify hospital cases into various groups. MDS (Minimum Data Set) is a federally mandated process for clinical assessment of all residents in Medicare or Medicaid certified nursing homes and non-critical access hospitals with swing bed agreements. MDS is a component of the PDPM (Patient Driven Payment Model), which is a new case-mix classification system for classifying skilled nursing facility (SNF) patients in a Medicare Part A covered stay into payment groups under the SNF Prospective Payment System. Effective Oct. 1, 2019, the PDPM will replace the current SNF patient case-mix classification system, the Resource Utilization Group, Version IV (RUG-IV). Patient Driven Groups Model (PDGM), which supports the payment by CMS for home health care related services effective Jan. 1, 2020, is a combination of ICD-10 diagnosis coding, OASIS (Outcome and Assessment Information Set) data, and additional patient data to categorize payment periods into relevant payment category groups.

In this way, information about the wound, historical patient information, and/or historical wound information can be captured and entered into the system.

Server database 50 therefore catalogs and assesses the wound. Specifically, system 1 is designed to accept and automatically measure the wound directly from the digital image. The database therefore acts as a centralized repository of the data stored in the method and system of the present disclosure, such as patient information, medical history, current medications, recent illness, and wound information including but not limited to, length, width and depth measurements of the wound, as well as wound images.

At step 20, the image is reviewed by a professional. In some embodiments, the professional is a medical professional or expert in the field of wound treatment. Thus, the disclosed system can be used by credentialed physicians and certified wound care nurses to review the wounds and consult on the care treatment. Alternatively, the image can be reviewed by a medical professional who supports patient care but does not have certified wound care experience, such as a Registered Nurse, Physician's Assistant, and/or a Medical Aide.

In some embodiments, the image can be provided to the professional through the system mobile application. Further, the provider and/or payor managing the patient can be provided access to review patient care progress and/or outcomes.

At step 25, a treatment plan is recommended by the system. As shown in FIG. 5, any of a variety of factors are considered and/or calculated to determine a suitable treatment plan for a patient. For example, coding assignments, historical wound outcomes, historical wound profit/loss figures, historical quality scores, historical standardized treatment plan, historical formulary, treatment location, provider type, frequency of visits, aggregated data (YTD) wound outcomes, wound classification, aggregated data (YTD) wound economic data, aggregated data (YTD) quality scores, collaboration with other care providers, aggregated data (YTD) disqualification factors, dynamic formulary, treatment environment (e.g., location and/or dependencies), standardized treatment plans, aggregated data (YTD) wound outcomes, and/or historical disqualification data points can all be considered.

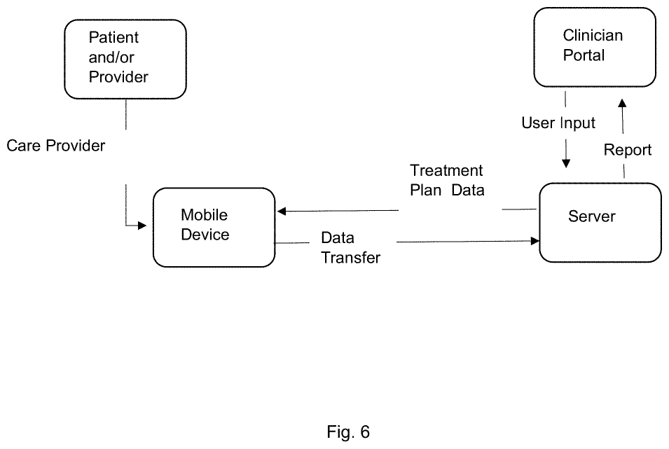

FIG. 6 illustrates a diagram illustrating one embodiment of a system flow process that can be used. As shown, an image of the wound is captured by imaging device 45, thereby generating image information that is transferred to the server. The server stores the data in a database together with previously generated wound parameters. The clinician can enter information about the wound and/or patient from a clinician portal to the server. Image and patient analysis is then performed on the transferred information. Display data and/or a treatment report can then be sent from the server to the patient and/or clinician.

Advantageously, the disclosed system includes the use of a combined data-driven, algorithmic, and machine learning approach to deliver optimal clinical care protocols and related wound care supply needs and written and video treatment instructions automatically at the point of care with a patient.

The term “algorithm” refers to any of a variety of statistical analyses used to determine relationships between variables. In the disclosed system, the variables are the coding assignments, historical wound outcomes, historical formulary, and the like. In some embodiments, logistic regression, linear regression, classification trees, and/or artificial neural networks (ANN) can be used. In some embodiments, a treatment plan can include a comparison of an index value for a wound to a threshold value for a particular characteristic or factor. A course of treatment can be determined based upon whether the index value is above or below the index cutoff value.

The term “logistic regression” refers to methods for predicting the probability of occurrence of an event by fitting data to a logistic curve. The term “linear regression” refers to attempts to model the relationship between two variables by fitting a linear equation to observed data. One variable is considered to be an explanatory variable, and the other is considered to be a dependent variable. The fitted (predicted) value of the response variable Y is a linear combination of the values of one or more predictor (X) variables. The term “classification tree” refers to one way of combining classification criteria into a tree structure so that after this connection objects may be placed in the tree automatically by comparing classification criteria in the tree branches with the classification data defined for the object. The term “artificial neural network” refers to any computational model that is capable of machine learning and pattern recognition. Artificial neural network types may vary from those with only one or two layers of single direction logic, to complicated multi-input many directional feedback loops and layers. One skilled in the art will understand how to employ such methods in the context of the disclosed system and method.

The disclosed system is also capable of machine learning techniques. Machine learning refers to the design and development of computer algorithms that allow computers to recognize complex patterns and make intelligent decisions based on empirical data. In the past, the indicators discussed above have been used in isolation to predict the response or lack thereof to a particular treatment. In comparison, the disclosed system combines the information from as many indicators/attributes as possible into a machine learning process which classifies the predicted patient diagnosis and/or response to a set of given treatments. The use of a wide assortment of features significantly improves the quality of the prediction in comparison to previous methods. As described above, the disclosed system can include a software application that can be integrated into any electronic medical record (EMR) application. For example, the application can be used to access the patient data to generate the optional clinical suggestions. Over time, the software application “learns” which clinical care options are selected most frequently by clinicians using the application and how often the clinical care selection increases the likelihood of a better patient outcomes. The information can be provided by multiple sources, such as subsequent wound measurement and/or patient health care status data available from patient care medical records in both acute (hospital, emergency, urgent care) and post-acute (skilled nursing, rehab facilities, and home care) settings.

The disclosed system therefore uses an algorithmic and machine learning approach to integrate selected data about a patient and/or wound and deliver clinical care suggestions immediately to the patient or caregiver. Information such as the patient's medical history and images of the patient wound are integrated to suggest optional clinical care protocols for the clinician to consider. The recommended treatment plan can include a standardized treatment plan and/or a formulary. The term “standardized treatment plan” refers to the normalized determination of an appropriate treatment for a patient's wound. The term “formulary” refers to a list of preferred pharmaceuticals and/or wound treatment materials to be used by medical professionals based on health care system and/or managed care plans (e.g. HMOs, ACOs) contracts with formulary item vendors, which are typically chosen based on the efficacy, safety, cost-effectiveness, and cost of the particular drug or material. System 5 therefore creates and maintains wound care protocols and supplies formularies to support efficient and effective patient wound care, and can be customized based on the system user formulary item preferences

Related wound care supplies can also be suggested to support the treatment protocol (e.g., through instructions and/or training videos that explain how the wound care supplies are used to deliver the expected wound care outcome). Typical formularies can include supply packs for varying degrees of chronic, non-healing, and traumatic wounds that are customized to treat a patient's specific wound. The formulary can thus include wound dressings, soaps, bandages, powder, compression sleeves, applicators, mattress overlays, saline, irrigants, syringes, sponges, ointments, creams, and the like. The wound supply packages can include written instructions on how to care for the wound and/or use the provided supplies, including system user options for language preferences (e.g. Spanish, Portuguese, Hebrew, etc). In some embodiments, the patient is notified when a treatment is to be performed, such as with a text message on the patient's phone or through the disclosed system. Multiple instructions can also be given to multiple parties. For instance, the health care professional can provide the patient's caregiver with verbal medical care instructions, a pharmacy with a prescription for filling, and a laboratory with advance notice that the patient will need a certain kind of test to be performed. The supply packages are therefore tailored to the wound location, stage of wound healing, wound care protocol and formulary, and/or duration of therapy, and language preferences for all written and audio communications

In some embodiments, formularies are available for delivery to clinics, skilled nursing facilities, or directly to patient homes.

The treatment plan therefore provides an easy to follow, comprehensive plan for healing the wound, customized for the particular patient and wound conditions. The treatment plan, including wound images and historical patient/wound information, can be accessible to both the patient and/or clinician. In addition, clinical discussions between a particular facility and a corresponding telehealth team can also be accessible to both the patient and clinician.

Progress of wound healing is then tracked and reported at step 30 of the disclosed method. Progress can be tracked through one or more factors, including (but not limited to) wound outcomes, wound profit/loss, and/or quality scores. The wound outcome refers to the ultimate conclusion at the end of the wound treatment. Factors can include whether the wound was healed, length of healing time, progress, and the like. The wound profit/loss factor can refer to the cost of treatment versus standard costs for admittance to either hospitals or hospital-based wound care centers and/or a hospital. Further, quality scores refer to the quantitative indication of the quality or severity of a respective wound measured at the onset and then during treatment. In some embodiments, the system can deliver daily/weekly/monthly summaries of clinical consultations, patient progress, wound care supplies used, heal rates, and the like to patient, providers, and/or payors managing a patient.

As set forth above, the disclosed system can include a mobile application. The mobile application can allow a user (e.g., patient and/or clinician) to directly view and/or upload information. For example, the application can include a secure connection, where each user must enter login and/or password information. In this way, all information is secure (e.g., HIPAA, HITECH compliant). The application can also allow the user to scroll through a list to find the correct patient. Alternatively, the user can search using patient information (date of birth, patient insurance ID, etc.), wound location, wound type, etc. The application can include a module that allows the wound location to be described using words, in addition to one or more diagrams that allow a user to graphically depict the wound location. For example, an image of the human body can be provided, allowing the user to select the hand (or other appropriate body part). An image of the selected body part can also be graphically provided, allowing the user to precisely include the wound location or other observations. In some embodiments, the application can periodically provide a series of descriptors to allow a user to accurately update or describe the wound (e.g., margin detail, drainage amount, drainage type, duration). In this way, progress of wound healing can be tracked by the system. At various time intervals, the system can send a user an update requesting additional information to be provided relative to the wound. For example, every 2 days the system can request an image of the wound be uploaded.

The application therefore provides a central database for all of the patient's information, including the treatment plan, instructional videos, and the like. In some embodiments, the app provides a module that allows the clinician to add notes about treatment. The app can include a portal to allow the patient to ask questions directly of the treating clinicians and/or any other professional.

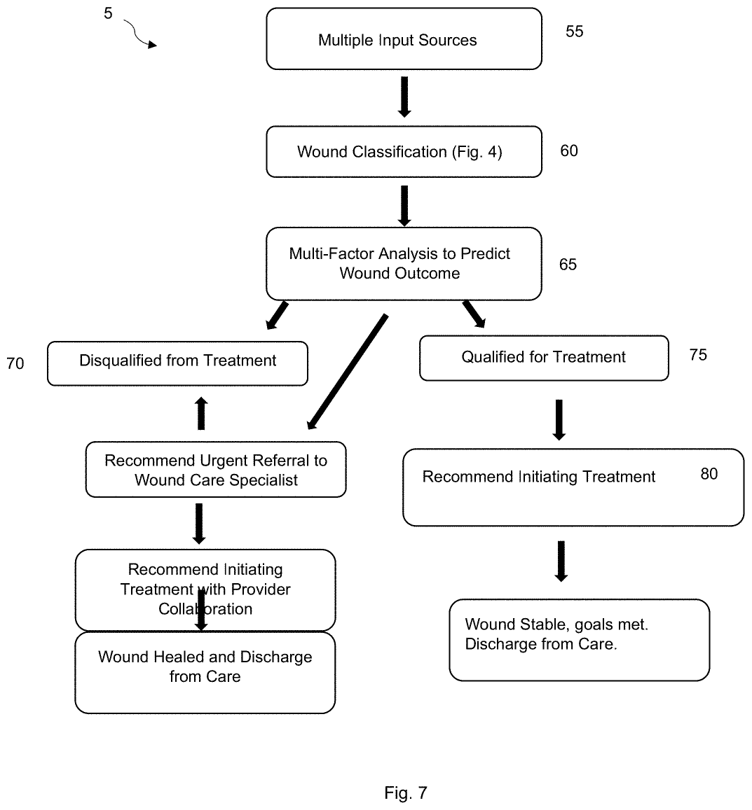

FIG. 7 is a flow chart illustrating one method of using system 1. At step 55, a clinician or patient can input the patient background and historical information into system 5, such as through the patient's EMR. A digital image of the wound is uploaded to the system by the clinician or patient using an imaging device. In some embodiments, the image can be captured by taking a photograph of the wound through a camera enabled by the system mobile application (e.g., from a smartphone or other imaging device). The information can be uploaded to the system using a mobile application, wirelessly uploaded, manually entered, and the like.

One or more code identifiers are automatically assigned to the wound at step 60 as the system factors the digital wound image, the patient history, the wound characteristics, etc. The codes provide information related to historical wound information, physical characteristics of the wound, etc. so that an outcome prediction can be made by the system at step 65. As shown, the patient is either disqualified or qualified for treatment based on the outcome prediction at steps 70, 75. If disqualified for treatment at step 70, the system provides either no further treatment plan information or provides recommendations for further diagnostic and/or therapeutic care, such as assessing arterial flow, treating diabetes, and the like. It should be appreciated that providing recommendations can lead to re-entry of the patient into the care pathway

If qualified for treatment, a treatment plan is recommended by the system at step 80. Specifically, the system uses an algorithm and/or machine learning to recommend a particular treatment, based on the inputs and the associated codes at treatment recommendation step 80. The recommended treatment can include steps for the patient to take, steps for the clinician to take, or both. For example, the patient may be instructed to wash the wound daily and apply antiseptic ointment. In other embodiments, the treatment can include the clinician debriding the wound, applying selected wound care dressings, and the like. After the treatment is recommended, a clinician reviews the recommended treatment at step 85 and determines whether the patient is disqualified from treatment at step 90 or moves to data collection step 95. The clinician can override the treatment plan or can proceed with the recommended treatment.

The presently disclosed subject matter is therefore generally directed to a system of effectively assessing and treating wounds. As described in detail above, the disclosed system allows for effective, efficient, and streamlined treatment of wounds through the use of a telehealth platform. Advantageously, the disclosed system includes the use of a combined data-driven, algorithmic, and machine learning approach to deliver optional clinical care protocols and related wound care supply needs and instructions automatically at the point of care with a patient. As a result, expert clinical wound care assessment is delivered rapidly, which reduces medical costs and clinical inconsistencies typically associated with manual wound review and treatment. The disclosed system also improves the speed with which wound care treatment begins. In addition, the system allows the patient to remain in the lowest acuity care environment (and lowest cost), such as at home. The system reduces the frequency and total number of visits to a clinical care facility, thereby reducing travel requirements. Further, the system allows the care process to be enabled by one or more persons with a lower level of professional training, including the patient themselves (self-care). The system standardizes care, beginning with the option to pursue a least expensive but highest quality alternative. The system additionally emphasizes the timely resolution of open wounds (e.g., infection reduction). Accordingly, the disclosed system provides for efficient and accurate personalized wound care for a subject in need of treatment.

One example of using the disclosed system to provide treatment is illustrated in FIG. 8. Initially, the wound type can be selected from an ulcer, surgical site infection, burn, bite, and the like. An image of the wound is input into the system, allowing the system to assign one or more codes to the wound. The system algorithm then provides a treatment outcome based on the image. For example, for a pressure ulcer, the decision can be to maintain the ulcer or debride the ulcer, depending on the algorithm. If the decision to debride is recommended, the image can determine heavy drainage, no drainage, dry drainage, or moderate drainage. If there is no drainage, the system can recommend a moist or dry wound bed. Similarly, if moderate drainage is observed, the system can recommend good or poor granulation. In the case of poor granulation, a bad or good wound edge can be identified.

Therefore, the disclosed system comprises a telehealth wound care system that provides physicians, hospitals, home health nurses, skilled nursing facilities, and patients with clinical wound care consulting and/or treatment through smart phone (or other device) imaging and custom wound supply delivery. As a result, the system supports patients with personalized wound care, enabling them to heal quickly and more affordably when compared to standard wound treatment methods.

The disclosed method and system therefore track and monitor compliance to wound care treatment plans, change in wound condition over time, and time to heal. The system can collect and compile data and/or statistics on all of the patients' information and wound information, and selectively provide information regarding any aspect of the same. For example, the percentage of male patients with a wound receiving a specific treatment. The information will be used by the system to improve treatment plans for a given patient. It is also foreseen that the system may incorporate clinical algorithms to recommend changes and/or enhancements to the user-defined preventive intervention plan or treatment plans accordingly. Further, the system can process patient-contributed wound images and communications that can be checked to assess adherence to wound care instructions and to schedule care events for patients and/or their attending providers. For example, care givers can be asked on a scheduled basis by the system to take an image (e.g., photograph) of the patient's wound at critical steps during the treatment plan. After a particular step has been completed and verified by the system, the treatment plan can annotate when an intervention was performed and used to track quality of care at the patient level.

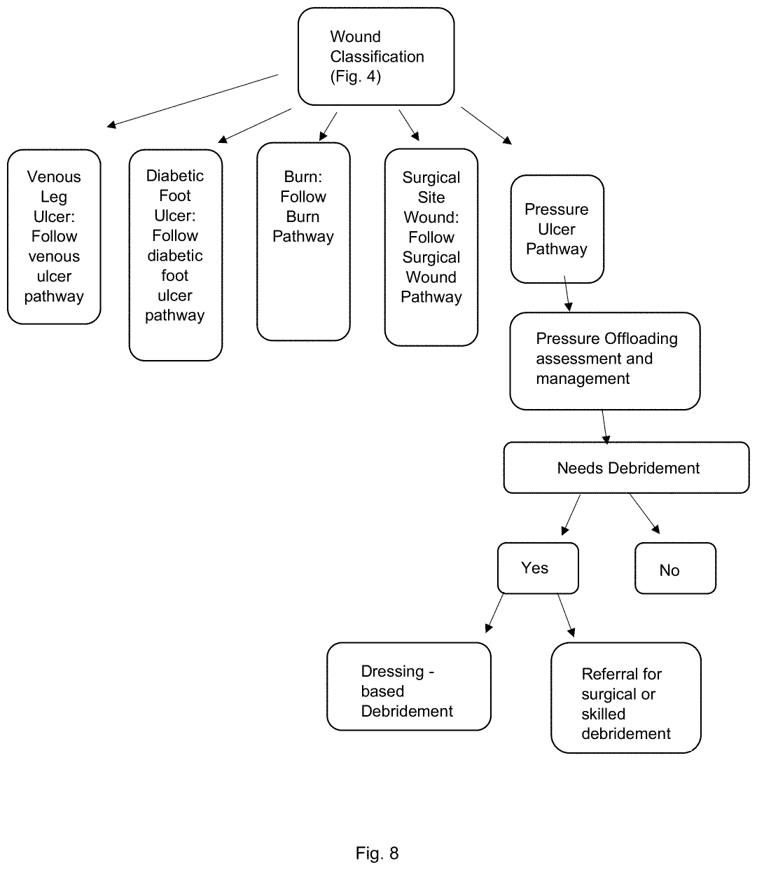

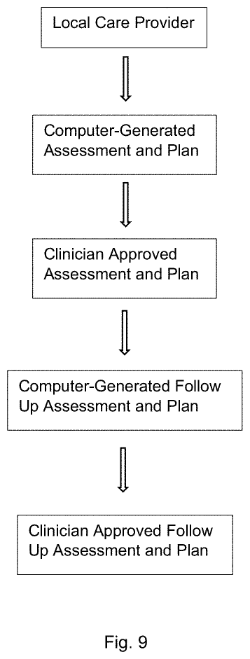

FIG. 9 is a flow chart illustrating one method of assessing a treating a wound in accordance with the presently disclosed subject matter. As a first step, a local care provider initiates a consult with the patient, including the provision of mandatory data related to the patient and/or wound. An image of the wound is then input into the disclosed system using an imaging device (e.g., a photograph of the wound is taken by the local care provider, a nurse, patient, or other individual).

A computer-generated assessment of the wound and a treatment plan are then provided by the system. The computer-generated assessment and plan can classify the wound, as well as provide an assessment of the caretaker and/or care environment. In some embodiments, the treatment plan can be managed by the disclosed system using input from the patient, caregiver, and/or clinician. Additional information may be required by the system to characterize the wound, characterize patient factors, and/or to assess the likelihood that the current patient environment can effectively support care. Initial treatment recommendations can include (but are not limited to) offloading the patient, debridement, anti-infective prescription, compression, dressing, and/or providing instructions to the caregiver. In some embodiments, the supply pack can be ordered to allow the patient or caregiver to begin basic wound care according to the treatment plan.

A qualified system clinician then reviews the computer-generated assessment and plan. The qualified clinician can optionally approve and/or edit the wound assessment and plan. Optionally, the qualified clinician can further prescribe medication as needed. Additionally, computer-learning feedback can be provided to the system.

After the qualified system clinician has approved the wound assessment and treatment plan, the information is input into the system. The wound is again classified and assessment of the caretaker and care environment are provided. Additional information may be required to initiate a customized prescription for the patient, further characterize the wound, patient factors, and/or the likelihood that the current patient environment can support care. An initial treatment prescription is provided, which can include offloading the patient wound (e.g. footwear, seat cushions, etc), debridement, anti-infective medications, compression devices, wound dressings, and/or instructions to the caregiver. In some embodiments, treatment supply packs can be ordered automatically and sent to the patient, along with supplies and instructions.

The system can track when supplies and instructions are sent to the patient, when medical record encounters are closed, and can provide communication back to the local care provider. Specifically, the local care provider can initiate the customized treatment plan, gather requested additional data (if any), provide additional images of the wound at requested intervals, make/receive queries regarding treatment, and/or provide images of treatment techniques. The provided caregiver skill assessment and images, wound interval images, and requested follow up clinical data are then analyzed by the system to optionally generate a computer-generated follow up assessment and plan.

The follow assessment and plan can include a reassessment of the wound environment, wound classification, wound progress, surrounding tissues/environment, host factors. In addition, an assessment of the caretaker skill and compliance may be generated. In some embodiments, a change in the care plan can be recommended.

A qualified wound care clinician can confirm the computer-generated follow up and assessment plan (e.g., approve and/or edit as needed). The qualified wound care clinician approved follow up assessment and plan can include a reassessment of the wound environment, wound classification, wound progress, surrounding tissues/environment, and host factors. In addition, assessment of the caretaker skill and compliance can be assessed. Prescribed changes in the care plan can also be input, which can include ordering and delivering supply packs. In some embodiments, ordering supply packs triggers sending the supplies to the patient, along with instructions for use.

Communication back to the local care provider can be provided, including initiation of customized treatment, gathering requested information, queries regarding treatment back to the system, providing wound images at requested intervals, providing images documenting treatment technique, and the like.

The term “patient” has been used primarily herein with reference to a human patient. However, the presently disclosed subject matter is not limited and can extend to any subject having a wound in need of treatment. Thus, in some embodiments, the disclosed system and method is suitable for veterinary use and can be used to treat dogs, cats, horses, and the like.

As set forth in detail above, the presently disclosed system provides many advantages. For example, system 5 can prevent or reduce the incidence of over or under-treating patients by providing tracked, personalized treatment plans.

The mobile application, wound review, clinical care protocols, and tailored wound care supplies support helping patients heal faster. As a result, patients and clinicians are able to manage wound care independently, saving time and money.

The disclosed system further supports providers with integrated telehealth wound care service through mobile triage and consult applications, helping patients heal faster and enabling them to manage their wound care independently.

Advantageously, the disclosed system is fully HIPAA compliant (i.e., cooperates with and follows the laws set forth in Congress in all three waves of HIPAA legislation).

The disclosed system is secure, requiring access credentials from users. In addition, all data is encrypted to protect patient confidentiality.

The disclosed system provides additional benefits for patients that cannot easily travel (e.g., inmates, disabled patients, patients without a vehicle, patients that live in rural locations). Specifically, the system can improve the speed of wound care treatment by providing access to certified wound care expertise without having to transport the patient to distant health care services locations.

The system allows users to store and forward wound images for streamlined and routine treatment care plans and share access to the patient wound images with medical professionals who are responsible for the patient care (e.g. primary care physicians, care management staff, etc.).

Advantageously, the system reduces the high cost of wound care by allowing for a reduction in transportation costs and security risks associated with high-risk patients (e.g., inmates).

The system reduces the number of emergency room visits and hospitalizations for acute and chronic wounds.

The system further improves patient and employee safety by providing unskilled onsite clinicians with access to certified wound care clinicians.

Patient outcomes are improved through timely and appropriate wound care. The system therefore reduces hospital readmissions for surgical site wound infections and allows for detection of surgical site wound infections earlier.

The system can identify outlier providers with a history of utilizing unnecessary services.

System 5 further provides improved quality metrics for patient care.

The disclosed system builds patient confidence with frequent wound care telehealth monitoring.

It should be understood that, while these exemplary systems include many different elements, which will be described in the sections that follow, various other embodiments of such system(s) may also include additional or fewer elements, as desired by the user and contemplated by the present disclosure.

### EXAMPLES

The following Examples provide illustrative embodiments. In light of the present disclosure and the general level of skill in the art, those of ordinary skill in the art will appreciate that the following Examples are intended to be exemplary only and that numerous changes, modifications, and alterations can be employed without departing from the scope of the presently disclosed subject matter.

### Example 1

### Forearm Wound Treatment

A female patient was admitted to a university medical center ER with a forearm laceration/avulsion injury from a domestic accident. The ER attending physician closed the forearm injury using stitches, and an orthopedics consult then cleared the patient for discharge to home without the provision of wound care instructions. As a result, the traumatized tissue became necrotic.

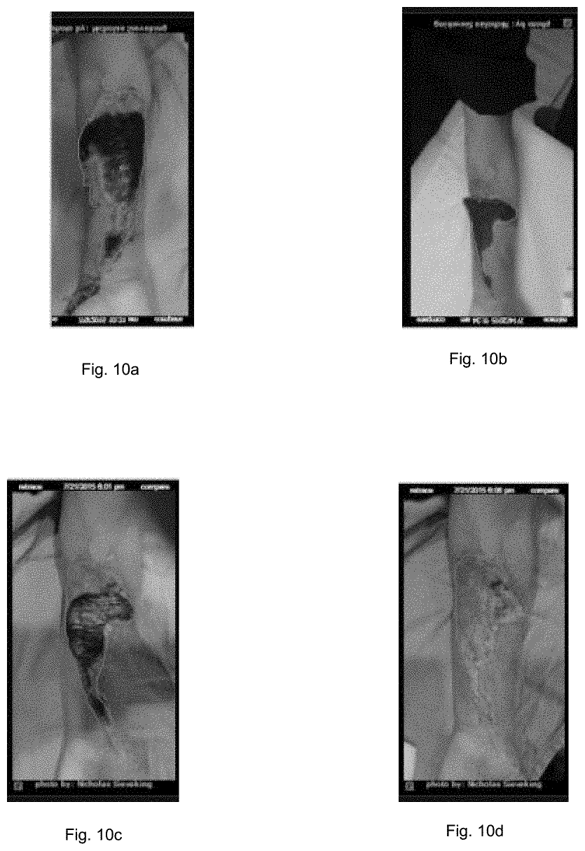

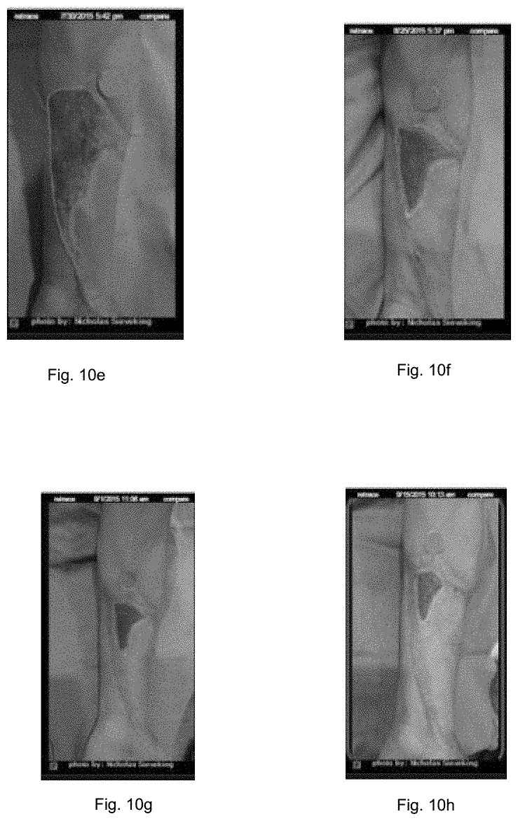

Outpatient wound care was then initiated using the disclosed system. For weeks 1 to 5, topical SulfaSilvadene cream was applied to the wound as a broad-spectrum antimicrobial and chemical debriding agent. For weeks 6 and 7, chloride solution was applied as a wet-to-wet dressing to debride the wound and decrease bacterial counts. Weeks 8 to 14, moist daily dressings were applied to the wound until full wound contraction and closure was achieved. FIGS. 10a-10h illustrate photographs of progression of wound healing throughout treatment. The wound was therefore successfully treatment at home by the patient using a treatment plan and supplies provided by the disclosed system.

### Example 2

### Animal Bite Wound Treatment

A male patient was seen in a hospital-based ER with an infected spider bite wound. The ER physician recommended inpatient care, surgical debridement, and broad spectrum intravenous antibiotic therapy.

Prior to beginning inpatient care, outpatient care using the disclosed system was initiated. The recommended care plan set included outpatient care with oral antibiotics (Augmentin®) and topical wound care comprising of Dial® soap and water. The treatment plan further included application of an antibiotic ointment covered with a moist dressing. Daily images of the wound were monitored by the system over 7 days until closure of the wound was achieved.

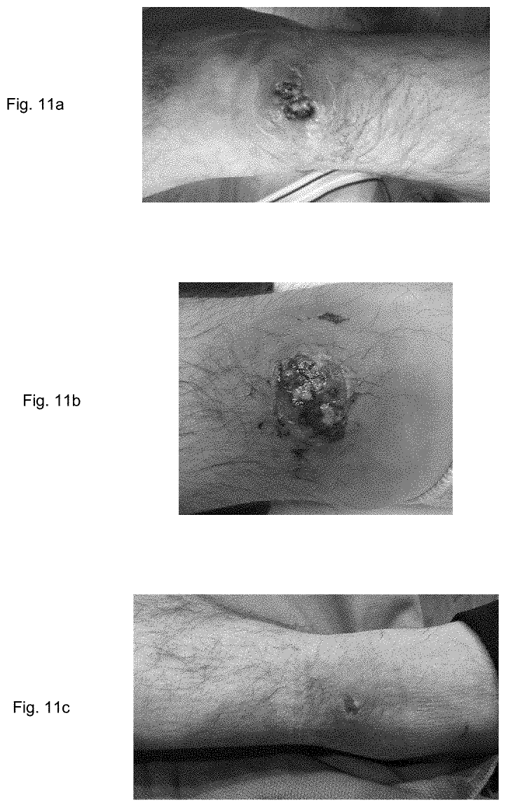

FIGS. 11a-11c illustrate progression of the wound healing during treatment. Accordingly, the wound was successfully healed using outpatient care with a treatment plan provided by the disclosed system.

### Example 3

### Surgical Site Infection Treatment

A patient was readmitted to a hospital with a surgical site infection from a recent spinal fusion procedure. The patient was admitted and placed on intravenous antibiotics (Vancomycin®) for 15 days. Multiple inpatient debridements were additionally performed. Due to lack of progress in wound closure, on hospital readmission day 15, the patient was discharged, and the disclosed treatment system was used.

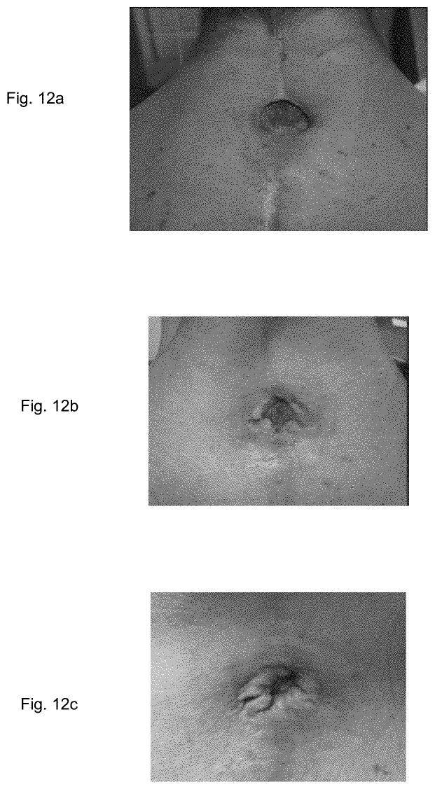

The treatment plan recommended the application of simple moist dressings and stopping the antibiotics. Instructions for the caretaker in performing the dressing changes was also provided. The wound completely healed in less than 2 weeks after discharge from the hospital, as illustrated in FIGS. 12a-12c.

### Example 4

### Hospital Pilot Study

The system was used as a pilot in a community-based Nashville hospital to measure the efficacy of using telehealth-supported clinical review of hospitalized patients to address surgical site infections (SSIs) and hospital-acquired pressure ulcers. At the hospital, the SSI rate was 9.8%. Hospital data estimated the cost of an SSI at that particular institution ranged between $10,000 and $40,000 per occurrence in uncompensated care. The figure was from 2010 data and did not include re-admission penalties.

The hospital pilot included 5 patients with SSIs and 1 patient with an acquired pressure ulcer from the hospital stay. After treatment using the disclosed system, efficacy was 100%, with no readmission and no hospital care (in patient or outpatient) after primary discharge. The conservative estimate of cost savings for the hospital was in excess of $135,000 in avoided hospital care expenses.

### Example 5

### Measurement Study

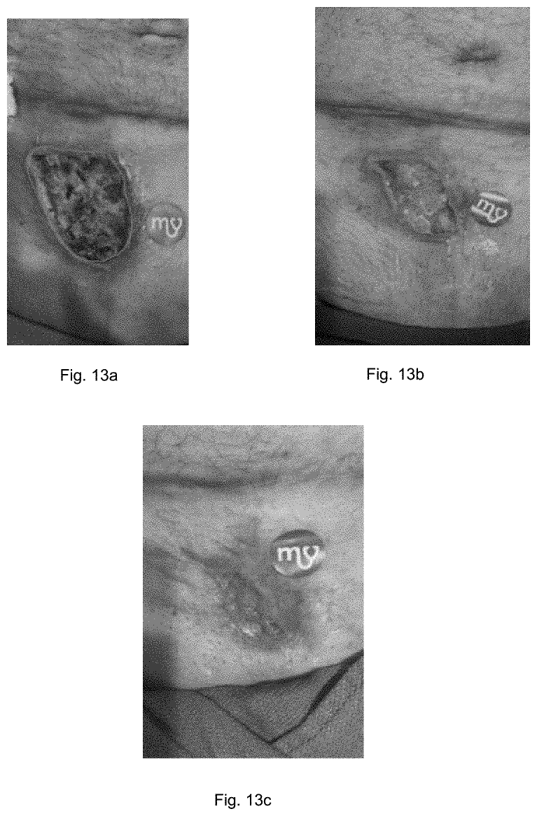

A patient with an open wound was selected to upload wound images during use of the system and recommended treatment protocols. The images are shown in FIGS. 13a-13c.

The disclosed system was able to capture the total area, length, width, and perimeter of the wound, as well as the total area of red, black, yellow, and “other” colors. The data was compared to initial data points in the initial photos to allow the user and treating clinician to track wound healing progress, as shown in Tables 1 and 2. The system therefore allowed the clinician to mark the wound as healed when applicable.

TABLE 1

| Measurement | Current | Vs. Initial | Change (%) |
|---|---|---|---|
| Total Area (cm²) | 14.69 | 25.29 | −42 |
| Length (cm) | 6.53 | 7.23 | −10 |
| Width (cm) | 3.15 | 4.69 | −33 |
| Perimeter (cm) | 8.15 | 9.88 | −14 |

TABLE 2

| Tissue | Area (cm²) | Area (%) | Vs. Initial | Change (%) |
|---|---|---|---|---|
| Red | 9.74 | 66 | 23.7 | −59 |
| Black | 0 | 0 | 0 | 0 |
| Yellow | 4.94 | 34 | 1.59 | +211 |
| Other | 0 | 0 | 0 | 0 |

What is claimed is:

1. A method for assessing a patient wound and providing a treatment plan, the method comprising:
inputting data into a database, wherein the data includes patient medical history information and an image of the wound;
correlating the data to a unique code to assess the wound;
determining qualification for treatment based on the assessment of the wound;
retrieving and storing collected data relating to historical wound assessment to provide a treatment plan for the wound; and
tracking and reporting progress of the treatment of the wound;
wherein the method facilitates lowest skilled care.

2. The method of claim 1, wherein the image of the wound is input into the database by uploading from an imaging device.

3. The method of claim 2, wherein the imaging device is selected from a camera-enabled mobile phone, smart phone, laptop computer, notebook computer, desktop computer, personal digital assistant, MP3 player, camera, ager, portable gaming device, electronic watch, portable music player, dedicated hardware computing device, or combinations thereof.

4. The method of claim 1, wherein the wound assessment comprises a determination of one or more of the following: patient medical history, wound exudate, wound odor, wound secondary infection, wound width, wound length, wound depth, and wound outcome.

5. The method of claim 1, wherein the database communicates with a mobile application for uploading, storing, and providing information related to the wound, the wound assessment, the treatment plan, and progress of the treatment of the wound.

6. The method of claim 1, wherein providing a treatment plan comprises a combined data-driven, algorithmic, and machine learning approach to deliver optimal clinical care protocols and related wound care supply needs and instructions.

7. The method of claim 1, wherein the wound is selected from one or more of a venous ulcer, skin ulcer, diabetic ulcer, pressure ulcer, surgical site infection, burn, or bite.

8. The method of claim 1, wherein facilitating lowest skilled care comprises providing treatment instructions and measuring compliance with treatment instructions.

9. The method of claim 1, wherein the treatment plan comprises instructions for healing the wound, procedures for healing the wound, formulary supplies, or combinations thereof.

10. The method of claim 1, further comprising shipping formulary supplies directly to the patient based on the treatment plan.

11. The method of claim 1, wherein the unique code comprises patient demographic data, patient medical history, wound characteristics, or combinations thereof.

12. A system for assessing and treating wounds in a patient, the system comprising:
an electronic device comprising a computer hardware, software, and display device for entering data about a patient into a database, wherein the data comprises patient medical history and an image of the wound;
wherein the computer hardware includes a storage device for receiving and storing information from the electronic device into the database;
a mobile application associated with the electronic device and the storage device for correlating the data to a unique code to assess the wound and determine qualification for treatment based on the wound assessment;
wherein the mobile application retrieves and stores collected data relating to historical wound assessment from the database to provide a treatment plan for the wound; and
wherein the mobile application provides for tracking and reporting progress of the treatment of the wound.

13. The system of claim 12, wherein the mobile application provides for the upload and display of an image of a wound.

14. The system of claim 12, wherein the image of the wound is input into the database by uploading from an imaging device comprising the mobile application.

15. The system of claim 14, wherein the imaging device is selected from a camera-enabled mobile phone, smart phone, laptop computer, notebook computer, desktop computer, personal digital assistant, MP3 player, camera, ager, portable gaming device, electronic watch, portable music player, dedicated hardware computing device, or combinations thereof.

16. The system of claim 12, wherein the wound assessment comprises a determination of one or more of the following: patient medical history, wound exudate, wound odor, wound secondary infection, wound width, wound length, wound depth, and wound outcome.

17. The system of claim 12, wherein providing a treatment plan comprises a combined data-driven, algorithmic, and machine learning approach to deliver optimal clinical care protocols and related wound care supply needs and instructions.

18. The system of claim 12, wherein the wound is selected from one or more of a venous ulcer, skin ulcer, diabetic ulcer, pressure ulcer, surgical site infection, burn, or bite.

19. The system of claim 12, wherein the mobile application coordinates shipping of formulary supplies directly to the patient based on the treatment plan.

20. The system of claim 12, wherein the unique code comprises patient demographic data, patient medical history, wound characteristics, or combinations thereof.
